(19) United States
(12) Patent Application Publication
(10) Pub. No.: US 2017/0273815 A1
(43) Pub. Date: Sep. 28, 2017

(54) WAIST SUPPORTER STRUCTURE WITH AN AUXILIARY BELT

(71) Applicant: FENG CHING TU, NEW TAIPEI CITY (TW)

(72) Inventor: FENG CHING TU, TAIPEI CITY (TW)

(21) Appl. No.: 15/471,735

(22) Filed: Mar. 28, 2017

(30) Foreign Application Priority Data
Mar. 28, 2016 (TW) 105204283

(51) Int. Cl. A61F 5/02

(52) U.S. Cl. CPC A61F 5/02

(57) ABSTRACT

A waist supporter structure with an auxiliary belt includes a waist pad, two knee wraps installed to both sides of the waist pad respectively and including a first belt member and a second belt member fixed to the waist pad and extended from both sides of the waist pad respectively, and two auxiliary waist belt devices fixed to both sides of the waist pad and extended from the sides of the waist pad respectively and including a first waist belt member and a second waist belt member, and the waist belt members are wound around a user's waist and buckled in front of the user's abdomen, so that when the user leaves the seat, the user needs not to readjust the waist supporting structure for reusing the structure, so as to improve the convenience of use.

### FIELD OF THE INVENTION

The present invention relates to a support structure, and more particularly to the support structure that allows users to reuse a waist supporter without the need of re-adjusting the waist supporter after stopping using the waist supporter temporarily, so as to improve the convenience of use.

### BACKGROUND OF THE INVENTION

### Description of the Related Art

In modern medicine, pelvic misalignment is generally corrected and treated by using a waist supporter structure to assist patients in their daily life. Since pelvic misaligmnent may cause tense muscle fascia and physical discomfort and make a patient's life inconvenient, therefore medical prevention equipments are required to maintain a normal daily life. With reference to FIGS. 1 and 2 for a conventional waist supporter, the conventional waist supporter comprises a waist pad 90, and a longer first belt member 91 and a shorter second belt member 92 disposed on both sides of the waist pad 90 respectively. The first belt member 91 has a knee pad 93, so that the first belt member 91 and the knee pad 93 jointly form a knee wrap assembly, and the first belt member 91 has a male buckle 95 and a female buckle 94 disposed at the front section of the knee pad 93 and buckled with each other in order to fix the waist pad 90 at a user's waist position. In addition, the first belt member 91 has an adjustable buckle 96 bent and wound around the rear section of the knee pad 93, and the rear end of the adjustable buckle 96 is fixed to the shorter second belt member 92, and the adjustable buckle 96 includes a strap loop 961. After the first belt member 91 is passed and wound around the strap loop 961, an adjustable pulling belt 911 of the first belt member 91 is extended forward. When the waist supporter is used in a sitting position, the waist pad 90 is placed at the user's waist position first, and then the knee pad 93 is placed at the user's knee position, so that the knee wrap assembly is sheathed on the user's knee, and the adjustable pulling belt 911 of the first belt member 91 is adjusted until the knee wrap assembly is wound with the user's knee tightly, and the waist pad 90 is attached to the user's waist closely for supporting the user's pelvis and waist.

Although the aforementioned conventional waist supporter structure has the effect of supporting the user's pelvis and waist, it still has the following drawbacks. When the conventional waist supporter is used, the tightness is adjusted by the adjustable pulling belt 911. Specifically, the adjustable pulling belt 911 is pulled forward to adjust the knee wrap assembly to an appropriate tightness, but when the user is sitting, it is not ergonomic for the user to apply a force to pull the adjustable pulling belt 911 forward. Obviously, such operation is inconvenient, and it is difficult for the user to control the applying force accurately or adjust the knee wrap assembly to an appropriate tightness. If the adjustable pulling belt 911 is over-pulled, the user will need to perform the operations of releasing the first belt member 91 of the adjustable buckle 96 (or the strap loop 961) and pulling the adjustable pulling belt 911 forward again.

If the user needs to stop using the waist supporter structure for a while and leaves the seat, the user no longer needs to wear the structure or adjust the tightness of the knee wrap assembly again for a continual use.

In view of the drawbacks of the structural design of the conventional waist supporter, the inventor of the present invention based on years of experience in the related industry to conduct research and developed and finally designed an ergonomic, easily operated waist supporter structure with an auxiliary belt to overcome the drawbacks of the prior art.

Summary of the Invention

Therefore, it is a primary objective of the present invention to redesign a knee wrap assembly and an adjustable pulling belt to fit an ergonomic operation of a waist supporter and allow users to operate and apply force accurately.

Another Objective of the present invention is to provide a waist supporter structure users to reuse a waist supporter without the need of making readjustment after stopping the use of the waist supporter and leaving the seat temporarily, so as to improve the convenience, efficiency and accuracy of use.

To achieve the aforementioned and other objectives, the present invention provides a the present invention provides a waist supporter structure with an auxiliary belt comprising: a waist pad; two knee wraps, disposed on both sides of the waist pad respectively, and each knee wrap including a first belt member and a second belt member fixed to the waist pad and extended from the sides of the waist pad respectively; two knee pads, movably sheathed on the first belt member, and having a passing portion with a through channel, and the first belt member and the knee pad jointly forming a knee wrap assembly; two adjustable buckles, installed between the first belt member and the second belt member, and a front end of the adjustable buckle being fixed to the first belt member, and the second belt member being passed and installed to the adjustable buckle and wound backward to form a winding portion, and the winding portion causing the front end of the second belt member to form an adjustable pulling belt, so that the adjustable pulling belt may be movably pulled backward to adjust and operate the knee wrap assembly; two auxiliary waist belt devices, including first waist belt member and a second waist belt member fixed to both sides of the waist pad and extended from the sides of the waist pad respectively, and the first and second waist belt members are wound around a user's waist and buckled in front of the user's abdomen.

In the aforementioned structure, the first belt member is longer than the second belt member, and the first belt member and the second belt member are fixed to both sides of the waist pad respectively by a stitching portion.

In the aforementioned structure, the knee pad has a passing portion with a through channel, and the passing portion is formed by positioning and attaching upper and lower edges of a belt member to the knee pad.

In the aforementioned structure, the adjustable buckle includes at least one strap loop for passing and installing the second belt member.

In the aforementioned structure, the knee pad has a kneecap pad extended backwardly from the top of the knee pad.

The above and other objects, features and advantages of this disclosure will become apparent from the following detailed description taken with the accompanying drawings.

### BRIEF DESCRIPTION OF THE DRAWINGS

FIG. 1 is a schematic view of the structure of a conventional waist supporter;

FIG. 2 is a schematic view of the use of a conventional waist supporter;

FIG. 3 is a schematic view of a first preferred embodiment of the present invention;

FIG. 3a is a first partial blowup view of the first preferred embodiment of the present invention;

FIG. 3b is a second partial blowup view of the first preferred embodiment of the present invention;

FIG. 4 is a schematic view of the use of a first preferred embodiment of the present invention;

FIG. 5 is a schematic view of the structure of a second preferred embodiment of the present invention; and

FIG. 6 is a schematic view of the use of the second preferred embodiment of the present invention.

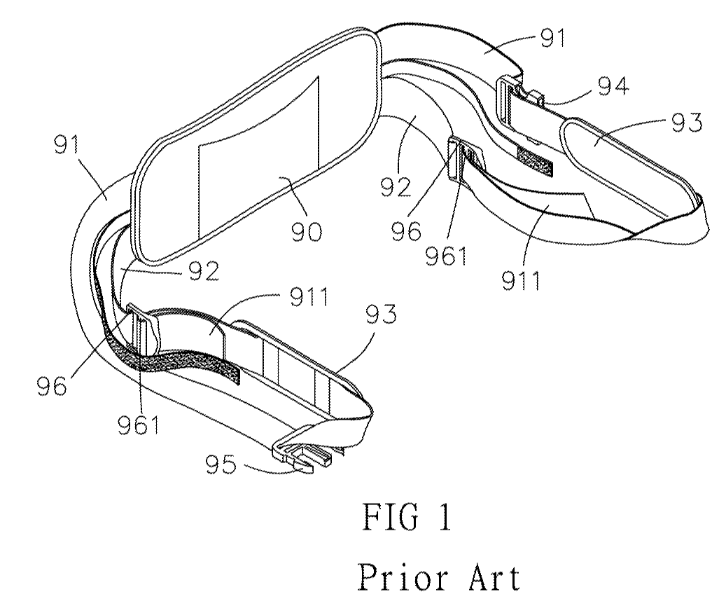

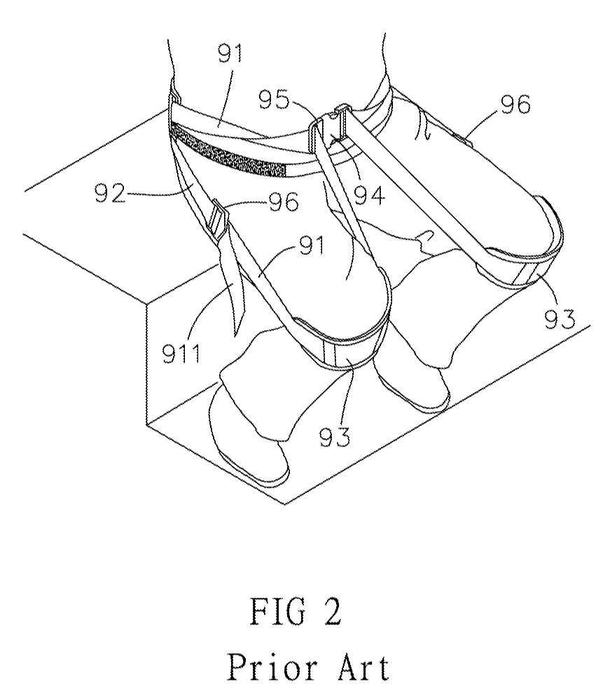

### DESCRIPTION OF THE PREFERRED EMBODIMENTS

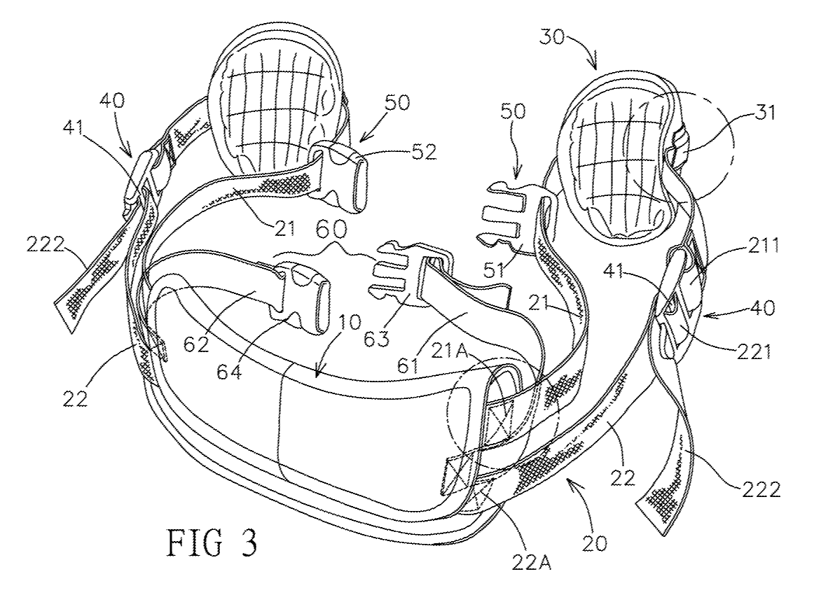

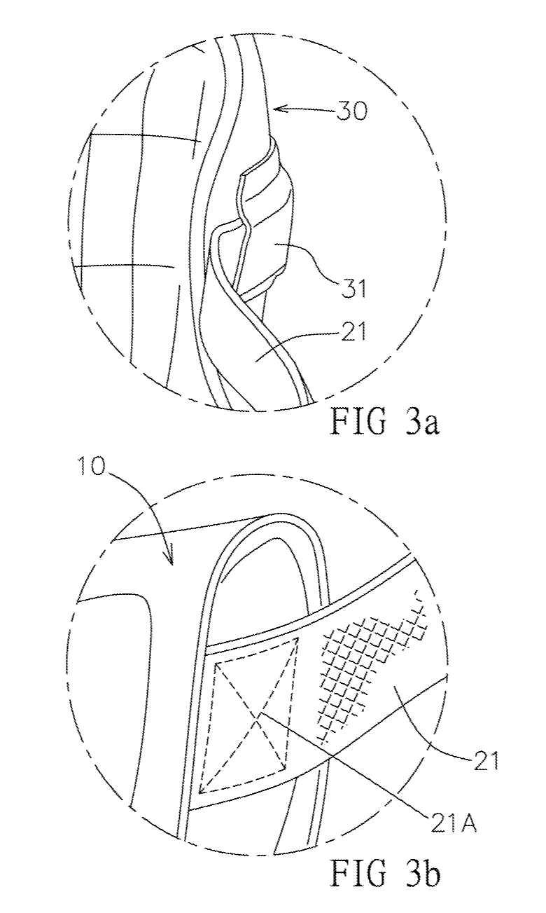

With reference to FIGS. 3, 3a, and 3b for a waist supporter structure with an auxiliary belt in accordance with the first preferred embodiment of the present invention, the waist supporter structure comprises a waist pad 10, two knee wraps 20, two knee pads 30, two adjustable buckles 40 and two auxiliary waist belt devices 60, wherein, the waist pad 10 may be a rectangular pad wrapped with a soft material (such as a sponge), and the outer surface of the waist pad 10 is made of a breathable fabric material to improve the comfort of use. The two knee wraps 20 are installed on both sides of the waist pad 10 respectively, and the two knee wraps 20 include a longer first belt member 21 and a shorter second belt member 22 fixed to the waist pad 10 and extended. from the sides of the waist pad 10 respectively as shown in the figures, and the first belt member 21 and second belt member 22 are fixed to the sides of the waist pad 10 by a stitching portion 21A and a stitching portion 22A respectively, and the first belt member 21 is usually installed at the top of the second belt member 22, but he invention is not limited to such arrangement only. The knee pad 30 is movably sheathed on the first belt member 21. In other words, the knee pad 30 has a passing portion 31 with a through channel, and the passing portion 31 may be formed by positioning and attaching upper and lower edges of a belt member, and the first belt member 21 is passed and installed to the passing portion 31, so that the knee pad 30 is sheathed and positioned onto the first belt member 21. When the knee pad 30 is used and covered onto a user's knee position, the first belt member 21 and the knee pad 30 jointly form a knee wrap assembly (not labeled in the figure).

The adjustable buckle 40 is installed between the first belt member 21 and the second belt member 22 and provided for adjusting the tightness of the knee wrap assembly, and the adjustable buckle 40 may be a loop buckle, but the invention is not limited to such design only. The front end of the adjustable buckle 40 is fixed to the longer first belt member 21. In other words, after the first belt member 21 is passed and installed to the rear end of the knee pad 30, a fixing end 211 is fixed to the adjustable buckle 40, and the adjustable buckle 40 has at least one strap loop 41 that allows user to install the second belt member 22 and wind the second belt member 22 backward to form a movable winding portion 221, and the winding portion 221 makes the front end of the second belt member 22 to form an adjustable pulling belt 222 that can be pulled backward.

The two auxiliary waist belt devices 60 are fixed to both sides of the waist pad 10 respectively, and the auxiliary waist belt device 60 includes a first waist belt member 61 and a second waist belt member 62 fixed to the waist pad 10 and extended from the sides of the waist pad 10 respectively, and the first waist belt member 61 and second waist belt member 62 are wound around a user's waist and buckled in front of the user's abdomen. The first waist belt member 61 and the second waist belt member 62 have a pair of auxiliary buckles disposed at an end away from the waist pad 10 and buckled with each other, and the auxiliary buckle includes an auxiliary male buckle 63 and an auxiliary female buckle 64.

In addition, the waist supporter structure with an auxiliary belt of the present invention further comprises a positioning buckle 50 including a male positioning buckle 51 and a female positioning buckle 52 disposed on the two first belt members 21 separately, and the male positioning buckle 51 or the female positioning buckle 52 is disposed between the waist pad 10 and the knee pad 30, and the male positioning buckle 51 and the female positioning buckle 52 are buckled with each other, so that the first belt member 21 (or the waist supporter) can be hanged and positioned at the position of the user's waist.

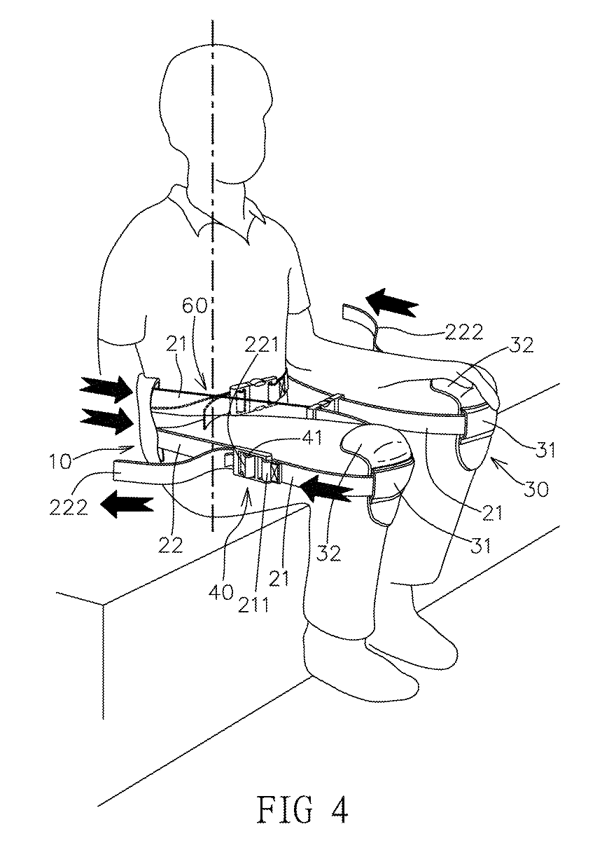

With reference to FIG. 4 for the using status of the waist supporter structure with an auxiliary belt in accordance with the present invention, the waist pad 10 is positioned at the user's waist first and then the first waist belt member 61 and the second waist belt member 62 are wound around the user's waist and buckled in front of the user's abdomen, and the first belt member 21 and the second belt member 22 are coupled by the adjustable buckles 40, and the knee wrap assembly (including the first belt member 21 and the knee pad 30) is wound or sheathed on the user's knee and the knee pad 30 is covered onto the user's knee position, and the adjustable pulling belt 222 of the second belt member 22 is pulled backward for making adjustment, so that the adjustable pulling belt 222 (second belt member 22) pulls the first belt member 21 backward by the adjustable buckle 40, and the knee wrap assembly is wound tightly with the user's knee. In the meantime, the waist pad 10 is attached closely to the user's waist for supporting the user's pelvis and waist.

In the adjustment operation of the present invention, the shorter second belt member 22 is movably wound with the adjustable buckle 40 to form a backwardly extended assembly for adjusting and operating the adjustable pulling belt 222, so that the backward pulling operation is ergonomic, convenient and easily controllable. In addition, the adjustable pulling belt 222 can be pulled sensitively and accurately to provide a good adjusting effect, so as to achieve the effect of providing the best effect of an appropriate, comfortable and effective waist supporter.

When the user needs to leave the seat and stop using the waist support structure temporarily, the user no longer needs to remove the waist supporter since the auxiliary waist belt device is still wound around the user's waist or re-adjust the waist support structure for a reuse. The invention has the feature of high convenience, efficiency and accuracy.

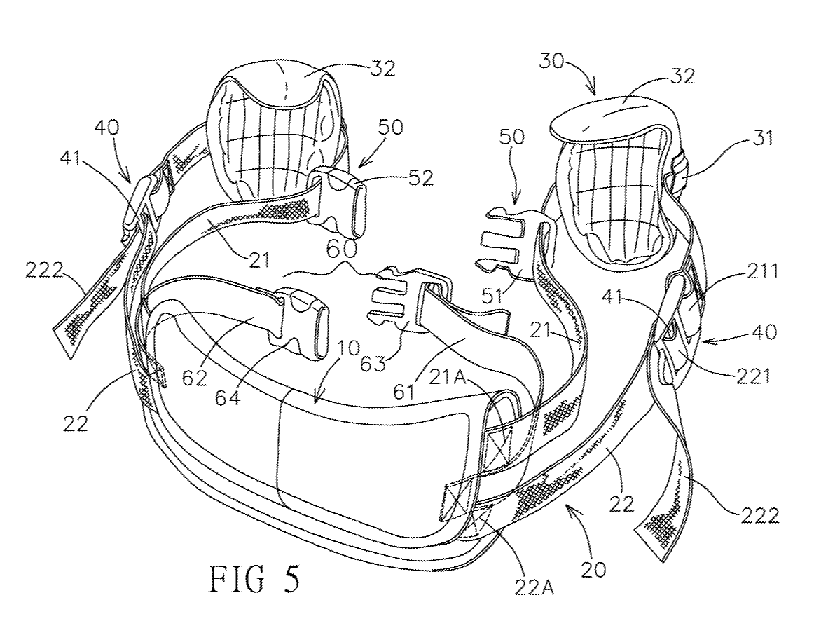

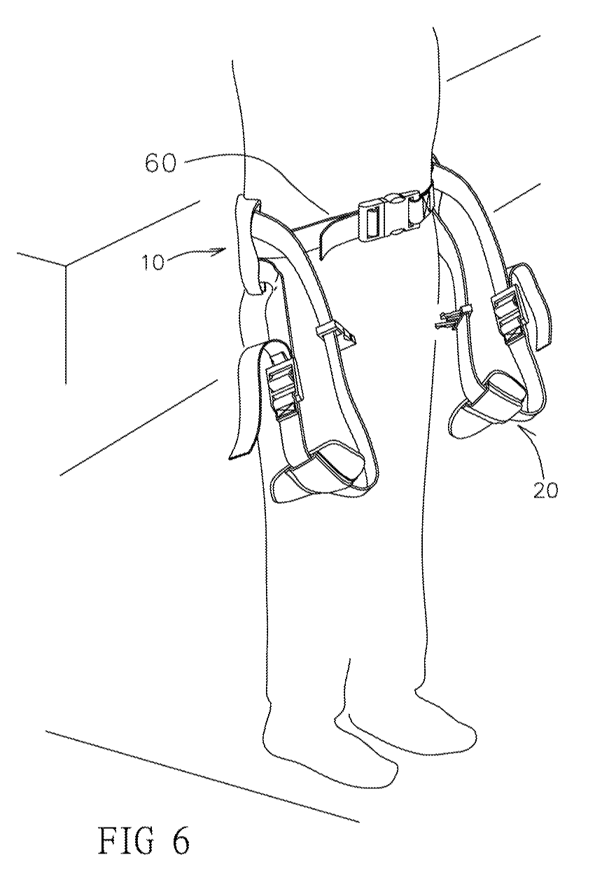

With reference to FIGS. 5 and 6 for a waist supporter structure with an auxiliary belt in accordance with the second preferred embodiment of the present invention, the structure of this embodiment is substantially the same as the first preferred embodiment, except that the knee pad 30 further comprises a kneecap pad 32 extended backward form the top of the knee pad 30 and capable of providing a better protective effect to the user's knee. In addition, the user may press on the kneecap pad 32 by a hand to achieve the effect of keeping the knee warm, and such arrangement is particularly good for a long time of use or a user with a weak knee joint.

In summation of the description above, the waist supporter structure with an auxiliary belt in accordance with the present invention meets the ergonomic design requirement by re-designing the knee wrap assembly and the adjustable pulling belt, so that the operation is convenient. When the user needs to leave the seat and stops using the waist support structure, the auxiliary waist belt device is still wound around the user's waist, so that the user needs not to remove the waist supporter, or re-adjust the waist supporter for a reuse. Therefore, the present invention has the effects of high efficiency and accuracy.

While the invention has been described by means of specific embodiments, numerous modifications and variations could be made thereto by those skilled in the art without departing from the scope and spirit of the invention set forth in the claims. In addition, the present invention complies with patent application requirements, and is thus duly filed for patent application.

What is claimed is:

1. An waist supporter structure with an auxiliary belt, comprising:
a waist pad;
two knee wraps, disposed on both sides of the waist pad respectively, and each knee wrap including a first belt member and a second belt member fixed to the waist pad and extended from the sides of the waist pad respectively;
two knee pads, movably sheathed on the first belt member, and having a passing portion with a through channel, and the first belt member and the knee pad jointly forming a knee wrap assembly;
two adjustable buckles, installed between the first belt member and the second belt member, and a front end of the adjustable buckle being fixed to the first belt member, and the second belt member being passed and installed to the adjustable buckle and wound backward to form a winding portion, and the winding portion causing the front end of the second belt member to form an adjustable pulling belt, so that the adjustable pulling belt may be movably pulled backward to adjust and operate the knee wrap assembly;
two auxiliary waist belt devices, including first waist belt member and a second waist belt member fixed to both sides of the waist pad and extended from the sides of the waist pad respectively, and the first and second waist belt members are wound around a user's waist and buckled in front of the user's abdomen.

2. The waist supporter structure with an auxiliary belt according to claim 1, wherein the first belt member is longer than the second belt member.

3. The waist supporter structure with an auxiliary belt according to claim 2, wherein the first belt member and the second belt member are fixed to both sides of the waist pad respectively by a stitching portion.

4. The waist supporter structure with an auxiliary belt according to claim 1, wherein the knee pad has a passing portion with a through channel.

5. The waist supporter structure with an auxiliary belt according to claim 4, wherein the passing portion is formed by positioning and attaching upper and lower edges of a belt member to the knee pad.

6. The waist supporter structure with an auxiliary belt according to claim 1, wherein the adjustable buckle includes at least one strap loop for passing and installing the second belt member.

7. The waist supporter structure with an auxiliary belt according to claim 1, further comprising a positioning buckle, and the positioning buckle including a male positioning buckle and a female positioning buckle installed to the two to first belt members respectively.

8. The waist supporter structure with an auxiliary belt according to claim 1, wherein the knee pad has a kneecap pad extended backwardly from the top of the knee pad.

9. The waist supporter structure with an auxiliary belt according to claim 1, wherein the first waist belt member and the second waist belt member have an auxiliary buckle at an end away from the waist pad, and the auxiliary buckles are bucklable with each other, and the auxiliary buckle includes an auxiliary male buckle and an auxiliary female buckle.
